(19) United States
(12) Patent Application Publication
(10) Pub. No.: US 2021/0325065 A1
(43) Pub. Date: Oct. 21, 2021

(54) METHOD AND APPARATUS FOR SUBSTANTIALLY INACTIVATING VIRAL REPLICATION IN FLOWING AIR

(71) Applicant: Michael Paul Kudinoff, Hacienda Heights, CA (US)

(72) Inventor: Michael Paul Kudinoff, Hacienda Heights, CA (US)

(21) Appl. No.: 17/354,961

(22) Filed: Jun. 22, 2021

Related U.S. Application Data
(60) Provisional application No. 63/042,701, filed on Jun. 23, 2020.

(51) Int. Cl. F24F 8/22; F24F 13/08; F24F 11/46

(52) U.S. Cl. CPC F24F 8/22; F24F 2110/30; F24F 11/46; F24F 13/08

(57) ABSTRACT

A method and apparatus for substantially deactivating viral replication in a flowing mass of air. According to the method implemented by the apparatus, viral replication is substantially inactivated by receiving a flowing mass of air, segregating the flowing mass of air into a plurality of channels and irradiating a portion of the segregated mass of air as it flows through the channel Light emitting diodes are used within each such channel to provide irradiance that is harmful to the reproductive structure of viruses, for example in a range between 240 nm and 300 nm.

### RELATED APPLICATIONS

The present application claims priority to U.S. Provisional Application Ser. No. 63/042,701, entitled “METHOD AND APPARATUS FOR SUBSTANTIALLY INACTIVATING VIRAL REPLICATION IN FLOWING AIR” by Kudinoff, which was filed on Jun. 23, 2020, the text and drawings of which are incorporated by reference into this application in their entirety.

### BACKGROUND

As the world know is emerging from a global pandemic, we have learned so much regarding our susceptibility to viral infection. We have learned, for instance, that confined spaces provide for easy transmission of a virus from one person to the next. In such confined spaces, air is typically circulated through a heating or air conditioning system.

All the most confined spaces do allow for “social distancing”, there are several environments where this is simply not possible. One such environment is an airliner. Especially in an economy class cabin, passengers are subject to close contact with strangers from around the world. This environment will continue to promote the spread of viral infections, such as Covid 19. And, because of the heating and air-conditioning ductwork in an airliner, viral matter is easily dispersed throughout the entire cabin. It is therefore essential to devise a means by which the recirculating error within the cabin is somehow cleansed from viral matter.

### BRIEF DESCRIPTION OF THE DRAWINGS

Several alternative embodiments will hereinafter be described in conjunction with the appended drawings and figures, wherein like numerals denote like elements, and in which:

FIG. 1 is a flow diagram that depicts one example method for inactivating viral replication in flowing air;

FIG. 2 is a flow diagram that depicts one alternative example method for receiving a flowing mass of air;

FIG. 3 is a flow diagram that depicts one alternative example method for segregating a mass of flowing air into a plurality of channels;

FIG. 4 is a flow diagram that depicts an alternative method for irradiating a portion of the segregated massive air;

FIGS. 5 and 6 are flow diagrams that depict alternative example methods for irradiating a flowing mass of air;

FIG. 7 is a flow diagram that depicts one alternative example method for reintroducing a massive flowing air back into a comfort volume;

FIGS. 8 and 9 are a pictorial illustrations of one example embodiment of an apparatus for inactivating viral replication in a flowing mass of air;

FIG. 10 is a pictorial illustration of one example embodiment of an irradiance air channel;

FIG. 11 is a pictorial illustration of one alternative example embodiments of an irradiance strip;

FIG. 12 is a pictorial diagram that illustrates one example embodiment of an irradiance strip that causes turbulent airflow within a channel; and

FIG. 13 is a block diagram that depicts one alternative example of a power control unit used to adjust the amount of irradiance applied to a flowing mass of air.

### DETAILED DESCRIPTION

In the interest of clarity, several example alternative methods are described in plain language. Such plain language descriptions of the various steps included in a particular method allow for easier comprehension and a more fluid description of a claimed method and its application. Accordingly, specific method steps are identified by the term “step” followed by a numeric reference to a flow diagram presented in the figures, e.g. (step 5). All such method “steps” are intended to be included in an open-ended enumeration of steps included in a particular claimed method. For example, the phrase “according to this example method, the item is processed using A” is to be given the meaning of “the present method includes step A, which is used to process the item”. All variations of such natural language descriptions of method steps are to be afforded this same open-ended enumeration of a step included in a particular claimed method.

Unless specifically taught to the contrary, method steps are interchangeable and specific sequences may be varied according to various alternatives contemplated. Accordingly, the claims are to be construed within such structure. Further, unless specifically taught to the contrary, method steps that include the phrase “ . . . comprises at least one or more of A, B, and/or C . . . ” means that the method step is to include every combination and permutation of the enumerated elements such as “only A”, “only B”, “only C”, “A and B, but not C”, “B and C, but not A”, “A and C, but not B”, and “A and B and C”. This same claim structure is also intended to be open-ended and any such combination of the enumerated elements together with a non-enumerated element, e.g. “A and D, but not B and not C”, is to fall within the scope of the claim. Given the open-ended intent of this claim language, the addition of a second element, including an additional of an enumerated element such as “2 of A”, is to be included in the scope of such claim. This same intended claim structure is also applicable to apparatus and system claims.

In many cases, description of various alternative example methods is augmented with illustrative use cases. Description of how a method is applied in a particular illustrative use case is intended to clarify how a particular method relates to physical implementations thereof. Such illustrative use cases are not intended to limit the scope of the claims appended hereto.

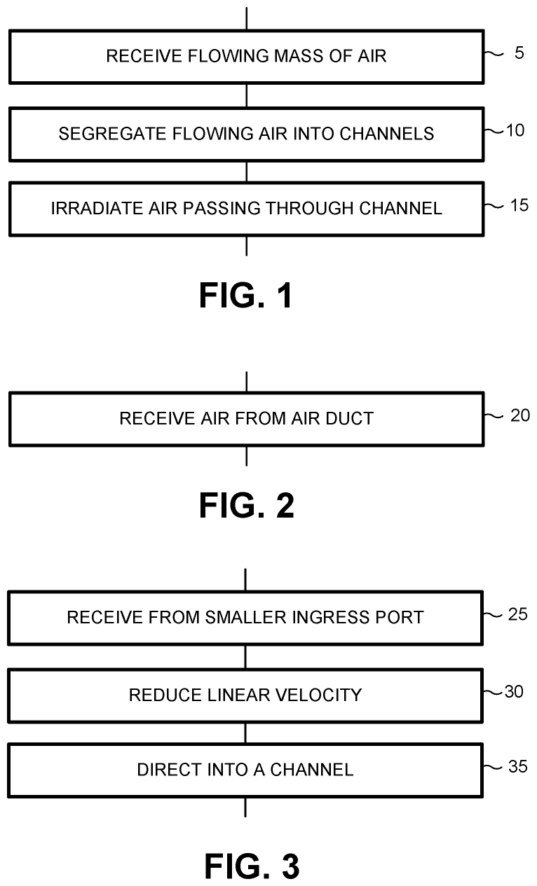

FIG. 1 is a flow diagram that depicts one example method for inactivating viral replication in flowing air. This example method comprises a step in which a mass of flowing air is received (step 5). An additional includes step provides for segregating the flow of air into a plurality of channels (step 10). This example method further includes a step four irradiating the air passing through the channel in order to deactivate any viral matter carried along with the flowing air.

FIG. 2 is a flow diagram that depicts one alternative example method for receiving a flowing mass of air. In this alternative example method, receiving a flow of air comprises receiving air from an air duct. It should be appreciated that air flowing through an airliner's cabin is channeled through ductwork. Although the descriptions you're in relates to ductwork in an aircraft, the claims appended hereto are not intended to be limited to any particular illustrative use case. For example, ductwork is also included in a residential home, and a commercial office building. Again these are merely illustrative use cases wherein the present method may be applied and are not intended to limit the scope of the claims appended hereto.

FIG. 3 is a flow diagram that depicts one alternative example method for segregating a mass of flowing air into a plurality of channels. In this alternative example method, on included step provides for receiving the flowing mass of air into a transition member, which is also known as an expander, in order to reduce the linear velocity of the air (step 30). It should be appreciated that, this alternative example method includes a step wherein the flowing mass of air is received into the transition member, wherein the transition member includes an ingress port and an egress port. In order to achieve reduction of the linear velocity of the air, the included method step provides for receiving error at an ingress port which is smaller in cross-section (step 25) than the egress port. The air is then directed into one of a plurality of channels (step 35) in an additional included method step.

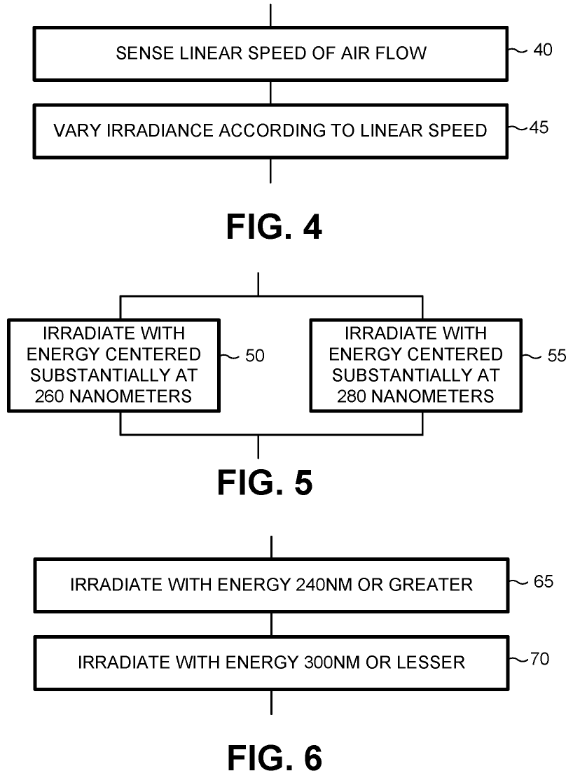

FIG. 4 is a flow diagram that depicts an alternative method for irradiating a portion of the segregated massive air. In this alternative example method, a step is included for sensing the linear speed of air flowing through channel (step 40) and then varying the radiance according to the linear speed (step 45). It should be appreciated that, according to various illustrative use cases, if the speed of the airflow is low, your radiance may be reduced. However, when the linear speed of airflow increases, so must the your radiance in order to allow for effective inactivation of viral matter carried by the flowing mass of air.

FIGS. 5 and 6 are flow diagrams that depict alternative example methods for irradiating a flowing mass of air. It should be appreciated that, according to several illustrative use cases, a flowing mass of air is irradiated by means of a light emitting diode (“LED”). Today, LEDs are available at a wide range of center wavelengths. It should be appreciated that all LEDs are substantially centered any particular wavelength. In order to be effective at inactivating viral replication, there appear to be two wavelengths of particular efficacy. According to one alternative example method, an included step provides for irradiating the flowing air with energies centered substantially at 260 nanometers (“nm”) (step 50). According to yet another alternative example method, an included step provides for irradiating the flowing air with energies centered substantially at 280 nm (step 55). And in yet another alternative example method, on included step provides for irradiating a portion of the segregated massive air with energy substantially within the range of 240 nm to 300 nm.

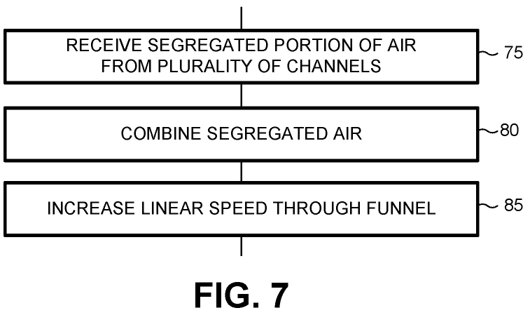

FIG. 7 is a flow diagram that depicts one alternative example method for reintroducing a massive flowing air back into a comfort volume. It should be appreciated that, according to this alternative example method, included steps provide for receiving segregated portions of air from a plurality of channels (step 75) and then combining the segregated air (step 80) into an amalgamated mass of flowing air. The linear speed of the flowing mass of air is then increased by directing the combined air through a funnel (step 85).

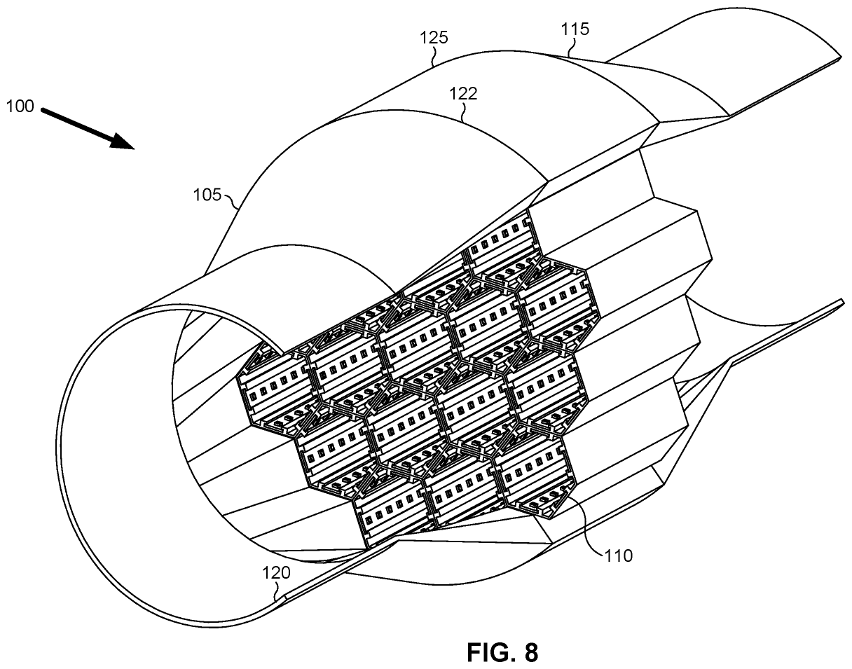

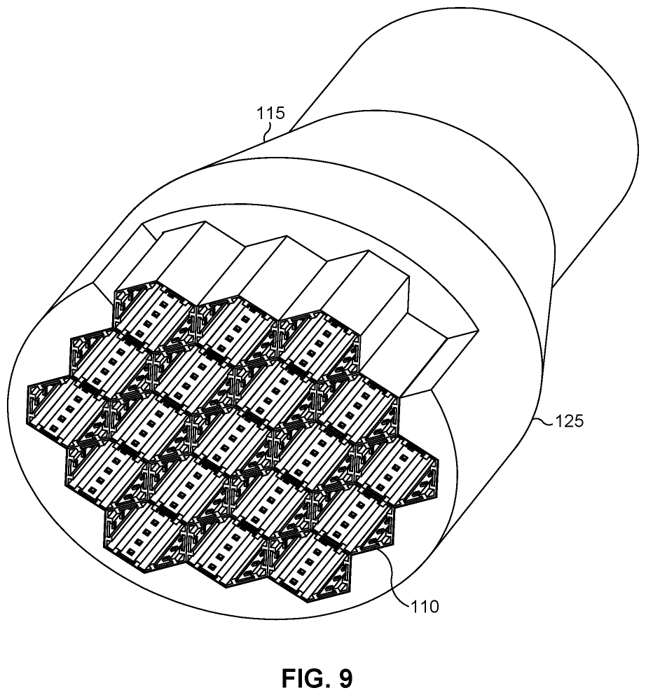

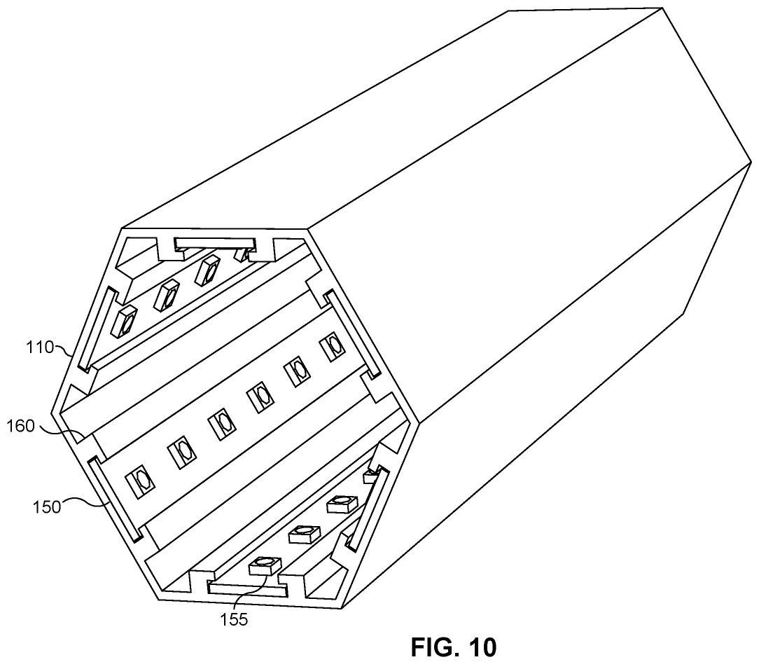

FIGS. 8 and 9 are a pictorial illustrations of one example embodiment of an apparatus for inactivating viral replication in a flowing mass of air. This example embodiment of the apparatus 100 comprises an expander 105. It should be noted that the expander 105 includes an ingress port 120 which is smaller in cross-section then an included egress port 122. The expander of this illustrative example embodiment causes the speed of air flowing therethrough to be reduced. This example embodiment of the apparatus 100 further includes a plurality of irradiance air channels 110. The each of these irradiance air channels receives a portion of the massive air flowing through the expander 105. Also included in this embodiment 100 is a funnel 115, which receives the airflow from the plurality of irradiance channels and re-combines them into a single flow of air.

FIG. 10 is a pictorial illustration of one example embodiment of an irradiance air channel. In this example embodiment, the air channel 110 includes one or more irradiance strips 150. In this example embodiment, the air channel comprises a tubular channel, which according to alternative example embodiments comprises at least one or more of a circular tube and/or a polygon 02. As depicted in the figures, this alternative example embodiments comprising a hexagonal channel. Each such air channel further comprises a support 160 which is used to retain and hold in position one or more irradiance strips 150. It should be appreciated that, according to various alternative example embodiments, the length of the channel depends on airflow and also depends on the radiated power provided by each LED used to provide irradiance within the channel 110.

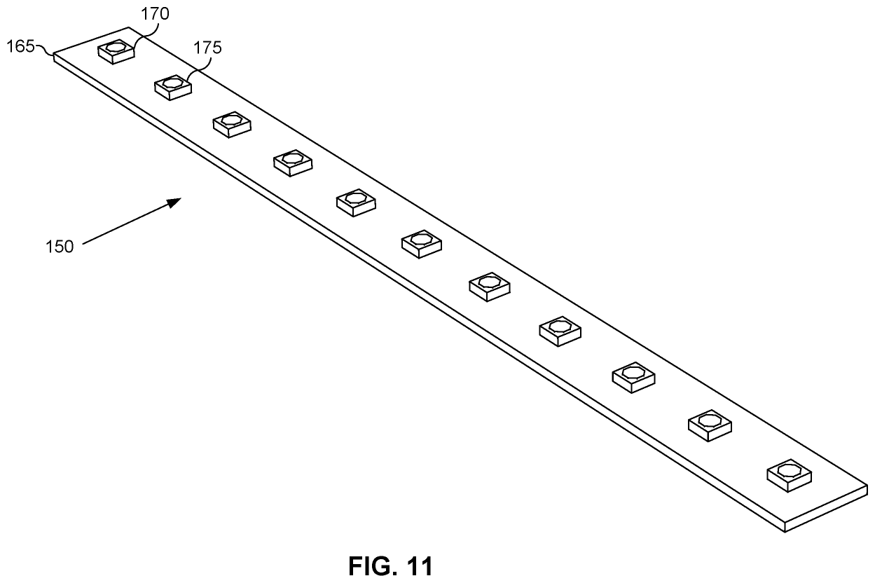

FIG. 11 is a pictorial illustration of one alternative example embodiments of an irradiance strip. According to this alternative example embodiment, these irradiance strip 150 includes a substrate 165. Also included in this alternative example embodiment of irradiance strip or a plurality of LEDs mounted upon the substrate 165. It should be noted that, according to yet another alternative example embodiment, the radiance strip 150 includes LEDs at varying wavelengths. In one alternative example embodiment the substrate 165 has mounted thereon an LED substantially centered at 260 nm 170. In yet another alternative example embodiment, the substrate 165 has mounted thereon an LED substantially centered about 280 nm. Is also important to note that, according to various alternative example embodiments, the LEDs included on the substrate 165 emits wavelengths of light substantially within a range of 240 nm through 300 nm.

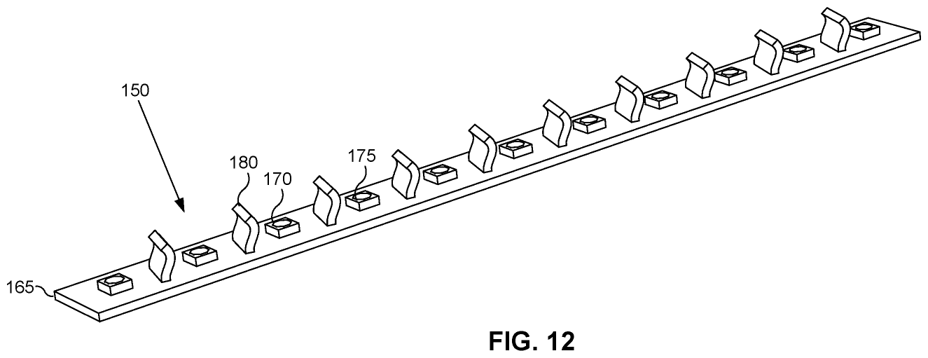

FIG. 12 is a pictorial diagram that illustrates one example embodiment of an irradiance strip that causes turbulent airflow within a channel. In this alternative example embodiment, the irradiance strip 150 still includes the substrate 165, LEDs of various wavelengths 170 and 175 but also includes a turbulence fin 180. The turbulence fin 180 is mounted on the irradiance strip 165 in order to agitate the flow of herein flowing through the air channel 110. Various shapes of turbulence fins 180 are herein contemplated.

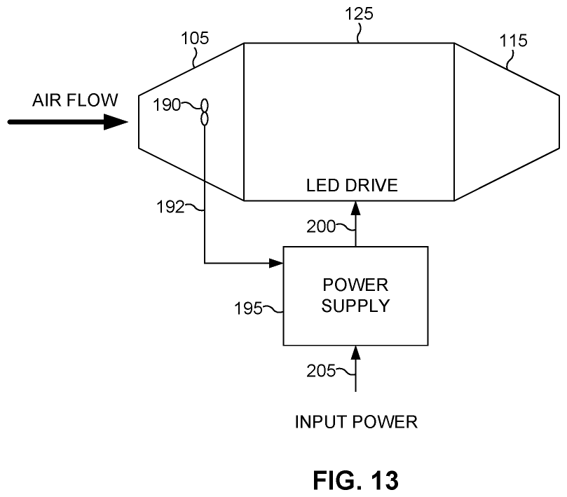

FIG. 13 is a block diagram that depicts one alternative example of a power control unit used to adjust the amount of irradiance applied to a flowing mass of air. As depicted in the figures, the apparatus 100 includes an expander 105, a channel chamber 125 and a funnel 115. Disposed within the expander is on included airflow sensor 190. The airflow sensor 190 generates an airspeed signal 192, which is directed to a power supply 195. The power supply 195 is configured to accept the airspeed signal 192 and to generate an LED drive signal 200 that is proportional to the airspeed indicated by the airspeed signal 192. Such control is known in the art and shall not be described herein.

The power supply 195 is also further configured to accept input power 205. Various forms of input power 205 or contemplated by a the disclosures herein.

While the present method and apparatus has been described in terms of several alternative and exemplary embodiments, it is contemplated that alternatives, modifications, permutations, and equivalents thereof will become apparent to those skilled in the art upon a reading of the specification and study of the drawings. It is therefore intended that the true spirit and scope of the claims appended hereto include all such alternatives, modifications, permutations, and equivalents.

What is claimed is:

1. A method for inactivating viral replication in flowing air comprising:
receiving a flowing mass of air;
segregating the flowing mass of air into a plurality of channels; and
irradiating a portion of the segregated mass of air as it flows through a channel.

2. The method of claim 1 wherein receiving a flowing mass of air comprises receiving a mass of air from an air duct.

3. The method of claim 1 wherein segregating the flowing mass of air into a plurality of channels comprises:
receiving the flowing mass of air into a transition member that includes an egress port having a cross section larger relative to an ingress port cross section;
causing the flowing mass of air to reduce its linear velocity; and
directing the reduced velocity air flow into one of a plurality of channels.

4. The method of claim 1 wherein irradiating a portion of the segregated mass of air comprises:
sensing the linear speed of air flowing through a channel; and
vary the amount of irradiance according to the linear speed.

5. The method of claim 1 wherein irradiating a portion of the segregated mass of air comprises irradiating the segregated mass of air with energy substantially centered at at least one or more of 260 nanometers and/or 280 nanometers.

6. The method of claim 1 wherein irradiating a portion of the segregated mass of air comprises irradiating the segregated mass of air with energy substantially within a range of 240 nanometers to 300 nanometers.

7. The method of claim 1 further comprising:
receiving segregated portions of air from a plurality of channel;
combining the segregated portions of air through a funneling member; and
increasing the linear velocity of the combined portions of air.

8. An apparatus for substantially inactivating viral replication in flowing mass of air comprising:
expander that includes an ingress port and an egress port, wherein the ingress port is smaller than the egress port;
plurality of irradiance air channels disposed to receive a flow of air from the egress port included in the expander; and
funnel disposed to receive air flow from the plurality of irradiance air channels.

9. The apparatus of claim 8 wherein an irradiance air channel comprises:
irradiance strips each including one or more light emitting diodes; and
tubular channel that includes supports for retaining the irradiance strips.

10. The apparatus of claim 8 further comprising:
airflow sensor that generate a speed signal proportional to the speed of air flowing through the apparatus; and
power supply that receives input power and generates a drive signal for the one or more light emitting diodes according to the speed signal.

11. The apparatus of claim 9 wherein the one or more light emitting diodes comprise at least one or more of a light emitting diodes substantially centered at 260 nanometers and/or a light emitting diodes substantially centered at 280 nanometers.

12. The apparatus of claim 9 wherein the one or more light emitting diodes emit radiation at a wavelength between 240 nanometers and 300 nanometers, inclusive.
